(19) United States
(12) Patent Application Publication
(10) Pub. No.: US 2022/0240962 A1
(43) Pub. Date: Aug. 4, 2022

(54) ENDOSCOPE FORCEPS

(71) Applicant: KANEKA CORPORATION, Osaka-shi, Osaka (JP)

(72) Inventor: Nobuyuki SUZUKI, Okaya-shi (JP)

(73) Assignee: KANEKA CORPORATION, Osaka-shi, Osaka (JP)

(21) Appl. No.: 17/611,609

(22) PCT Filed: Feb. 20, 2020
(86) PCT No.: PCT/JP2020/006746

§ 371 Date: Nov. 16, 2021

(30) Foreign Application Priority Data
May 20, 2019 (JP) 2019-094434

(51) Int. Cl. A61B 17/29; A61B 1/018; A61B 17/28

(52) U.S. Cl. CPC A61B 17/29; A61B 2017/0034; A61B 17/2841; A61B 1/018

(57) ABSTRACT

Endoscope forceps contains: a coil member having a distal end and a proximal end and including a lumen, the coil member made of a metal and having flexibility; a tubular member including a lumen and disposed in the lumen of the coil member; a pair of forceps members disposed on a distal side relative to the coil member, and openable and closable; connection members respectively connected to portions on a proximal side of the pair of forceps members; and a stranded wire inserted through the lumen of the tubular member and including a connection portion connected to proximal end portions of the connection members, wherein the distal end of the coil member is located between a distal end of the tubular member and the connection portion at which each connection member is connected to the stranded wire.

### TECHNICAL FIELD

The present invention relates to endoscope forceps used mainly for the purpose of hemostasis during a surgery or a treatment in which an endoscope is used.

### BACKGROUND ART

Forceps which are a treatment instrument for gripping a tissue in the body for the purpose of pickup and hemostasis of the tissue are used in a treatment in which an endoscope is used. The forceps not only have a function of gripping a tissue in the body which is an object to be gripped, but also can be used as a high-frequency treatment instrument by connecting a high-frequency power supply to a gripping member which is provided at a tip portion of the treatment instrument. For example, Patent Document 1 discloses endoscope biopsy forceps including an insertion portion and an operation portion, the insertion portion including: a coil including a lumen; an outer tube coating the outer surface of the coil; an inner tube disposed so as to be advanceable and retractable through the lumen of the coil; and two operation wires disposed so as to be advanceable and retractable through a lumen of the inner tube.

### PRIOR ART DOCUMENTS

### Patent Documents

Patent Document 1: JP-A-2000-175928

### SUMMARY OF THE INVENTION

### Problems to be Solved by the Invention

During use of the endoscope forceps, air feeding processing or liquid feeding processing is sometimes performed in order to make it easy to observe a lesion site with an endoscope. In this case, the internal pressure of the body increases at the time of air feeding or liquid feeding, and there is a concern that at least either of a liquid for the liquid feeding and a body fluid such as blood enters the endoscope forceps and reversely flows to a hand side. In addition, in the forceps described in Patent Document 1, friction resistance is generated owing to contact between the outer surface of each operation wire and the inner surface of the coil, and an opening/closing operation and a rotation operation of the forceps may become difficult to perform. In particular, in a case where a stranded wire having high torque-transmission characteristics is used as the operation wire, projections and recesses are formed on the surface thereof owing to twisting of wires, and thus there is a concern about increase in the friction resistance between the stranded wire and the coil. Considering this, an object of the present invention is to provide endoscope forceps capable of preventing contact between a stranded wire and a coil member and inhibiting a liquid such as a body fluid from reversely flowing to reach a hand side.

### Solutions to the Problems

An endoscope forceps of the present invention that has solved the above problems comprising: a coil member having a distal end and a proximal end and including a lumen, the coil member being made of a metal and having flexibility; a tubular member including a lumen and disposed in the lumen of the coil member; a pair of forceps members disposed on a distal side relative to the coil member, and openable and closable; connection members respectively connected to portions on a proximal side of the pair of forceps members; and a stranded wire inserted through the lumen of the tubular member and including a connection portion connected to proximal end portions of the connection members, wherein a distal end of the tubular member is located on the distal side relative to the connection portion between each connection member and the stranded wire so as to be present on the proximal side relative to the distal end of the coil member. By setting the position of the connection portion and the position of the distal end of the tubular member as described above, contact between the coil member and the stranded wire can be prevented. Thus, an opening/closing operation and a rotation operation of the forceps can be smoothly performed. In addition, disconnection between the connection member and the stranded wire due to contact of the coil member with the connection portion of the stranded wire, can also be prevented. Further, since the tubular member is disposed between the coil member and the stranded wire, a liquid such as a body fluid is less likely to enter the lumen of the coil member, and the liquid can be inhibited from reaching a hand side. By setting the position of the distal end of the tubular member and the position of the distal end of the coil member as described above, a distal end portion of the tubular member is covered with the coil member. Thus, even if heat and the like are generated by a high frequency during use of the forceps, influence of the heat on the tubular member can be mitigated.

The tubular member is preferably made of a synthetic resin. On the other hand, the stranded wire is preferably made of a metal.

A proximal end of the tubular member is preferably located on the proximal side relative to the proximal end of the coil member. Further, a proximal end portion of the tubular member is preferably fixed to the stranded wire.

Each connection member is preferably a single wire.

In a cross section of a distal end of the stranded wire perpendicular to a longitudinal direction of the coil member, a spatial cross-sectional area between the coil member and the tubular member is preferably larger than a spatial cross-sectional area between the tubular member and the stranded wire. It is also preferable that in a cross section of a distal end of the stranded wire perpendicular to a longitudinal direction of the coil member, a spatial cross-sectional area between the coil member and the tubular member is smaller than a spatial cross-sectional area between the tubular member and the stranded wire.

A distal end portion of the tubular member is provided with a smaller-diameter portion having an outer diameter that is preferably smaller than an outer diameter of the tubular member at a center position in a longitudinal direction thereof. Further, a distal end portion of the tubular member is preferably provided with a tapered portion that is tapered toward the distal side.

The endoscope forceps is preferable wherein a protection member connected to a proximal end portion of the coil member; and an operation member disposed on the proximal side relative to the coil member and configured to allow an opening/closing operation of the pair of forceps members to be performed through the operation member, wherein the operation member includes a first operation portion rotatably connected to a proximal end portion of the protection member, and a second operation portion connected to a proximal end portion of the stranded wire and configured to be moved relative to the first operation portion, and a connection portion between the protection member and the first operation portion is located on the distal side relative to a connection portion between the stranded wire and the second operation portion.

When the operation member is rotated with respect to the coil member, a rotation angle at the distal end of the tubular member is preferably smaller than a rotation angle at a proximal end of the tubular member.

### Effects of the Invention

By disposing the tubular member at the above predetermined position, contact between the coil member and the stranded wire can be prevented. Thus, an opening/closing operation and a rotation operation of the forceps can be smoothly performed. In addition, disconnection at the connection portion of the stranded wire due to contact between the coil member and the connection portion, can also be prevented. Further, a liquid such as a body fluid is less likely to enter the lumen of the coil member, and the liquid can be inhibited from reaching the hand side.

### BRIEF DESCRIPTION OF THE DRAWINGS

FIG. 1 is a side view of endoscope forceps according to an embodiment of the present invention.

FIG. 2 is an enlarged perspective view of a distal end portion of the endoscope forceps shown in FIG. 1.

FIG. 3 is an enlarged side view (partial cross-sectional view) of the distal end portion of the endoscope forceps shown in FIG. 1.

FIG. 4 is an enlarged cross-sectional view (partial side view) of a part on a proximal side of the endoscope forceps shown in FIG. 1.

### MODE FOR CARRYING OUT THE INVENTION

Hereinafter, the present invention will be more specifically described based on the following embodiments. However, the present invention is not limited to the following embodiments and, as a matter of course, can also be carried out with appropriate modifications being made within the scope of the gist described above and below, and any of these modifications are included in the technical scope of the present invention. In any of the drawings, hatching, reference characters for members, or the like may be omitted for convenience. In this case, see the description and the other drawings. Since priority is given to facilitating the understanding of the characteristics of the present invention, the dimensions of various members in the drawings may be different from actual dimensions.

Endoscope forceps according to an embodiment of the present invention include: a coil member including a lumen and having a distal end and a proximal end, the coil member being made of a metal and having flexibility; a tubular member including a lumen and disposed in the lumen of the coil member; a pair of forceps members disposed on a distal side relative to the coil member, and openable and closable; connection members respectively connected to portions on a proximal side of the pair of forceps members; and a stranded wire inserted through the lumen of the tubular member and including a connection portion connected to proximal end portions of the connection members, wherein a distal end of the tubular member is located on the distal side relative to the connection portion between each connection member and the stranded wire so as to be present on the proximal side relative to the distal end of the coil member. By setting the position of the connection portion and the position of the distal end of the tubular member as described above, contact between the coil member and the stranded wire can be prevented. Thus, an opening/closing operation and a rotation operation of the forceps can be smoothly performed. In addition, disconnection between the connection member and the stranded wire due to contact of the coil member with the connection portion of the stranded wire, can also be prevented. Further, since the tubular member is disposed between the coil member and the stranded wire, a liquid such as a body fluid is less likely to enter the lumen of the coil member, and the liquid can be inhibited from reaching a hand side. By setting the position of the distal end of the tubular member and the position of the distal end of the coil member as described above, a distal end portion of the tubular member is covered with the coil member. Thus, even if heat and the like are generated by a high frequency during use of the forceps, influence of the heat on the tubular member can be mitigated.

The endoscope forceps (hereinafter, sometimes referred to simply as forceps) are a treatment instrument to be inserted through a forceps channel of an endoscope and introduced into a body. The endoscope forceps hold a tissue in the body for the purpose of pickup and hemostasis of the tissue. In association with movement of the stranded wire to the distal side or the proximal side, the forceps members pivot with respect to each other, to become close to and apart from each other. Consequently, the forceps are opened or closed, and a tissue at a desired position in the body such as a submucosa tissue can be picked up. In addition, if a high-frequency power supply is connected to the forceps, the forceps can be used also as high-frequency hemostasis forceps.

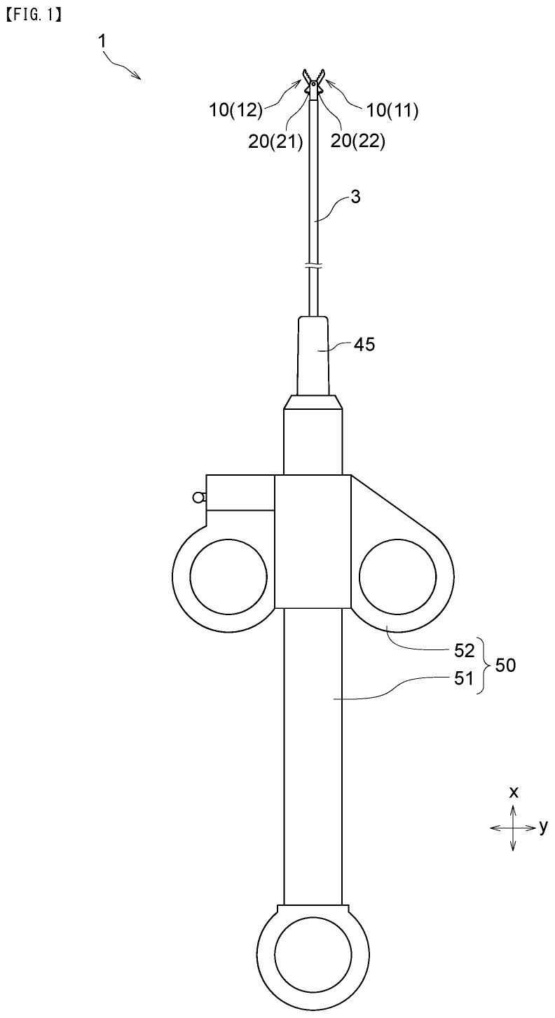

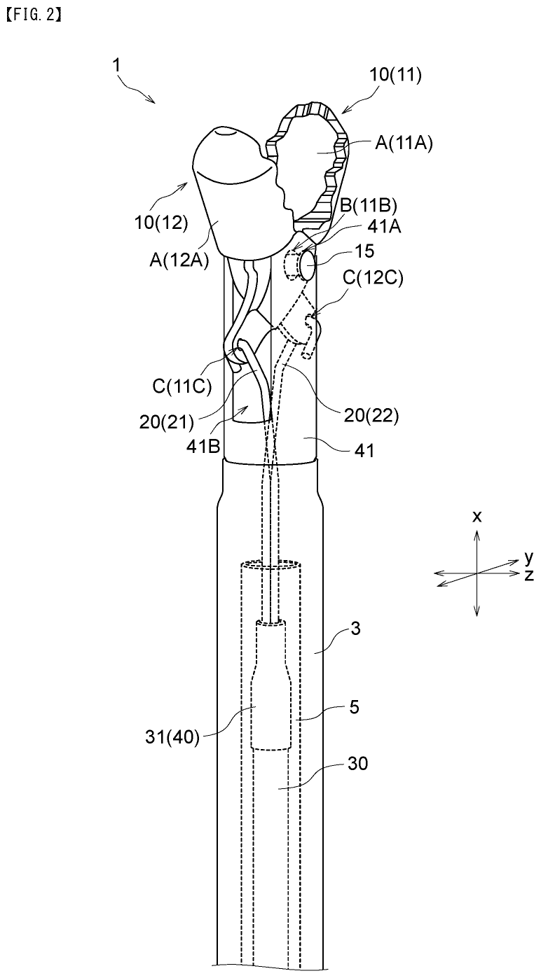

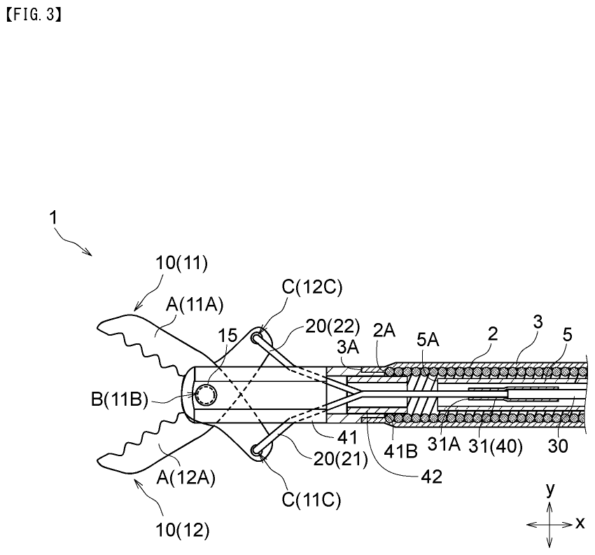

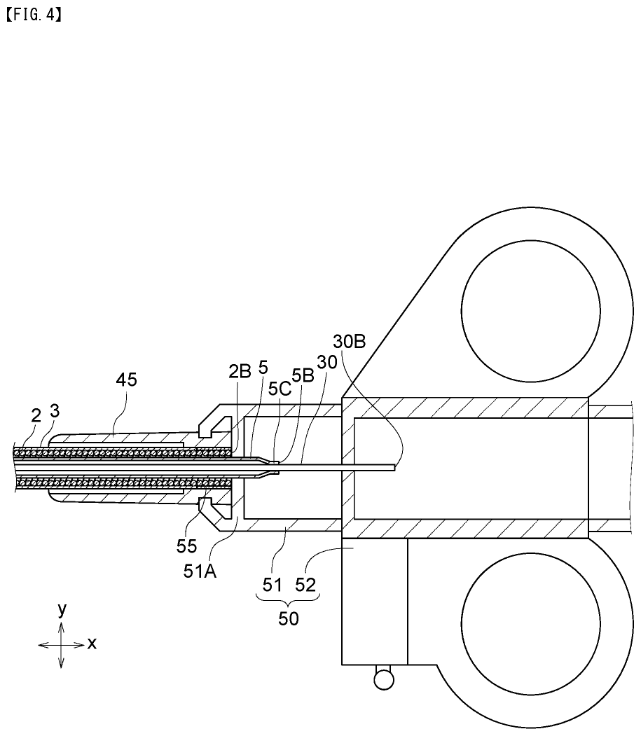

A configuration of the forceps will be described with reference to FIG. 1 to FIG. 4. FIG. 1 is a side view of the endoscope forceps according to an embodiment of the present invention. FIG. 2 and FIG. 3 are respectively an enlarged perspective view and an enlarged side view (partial cross-sectional view) of a distal end portion of the endoscope forceps in FIG. 1. FIG. 4 is an enlarged cross-sectional view (partial side view) of a part on the proximal side of the endoscope forceps shown in FIG. 1.

In forceps 1, the proximal side refers to a hand side of a user, i.e., an operator, in a longitudinal direction of a coil member 2, and the distal side refers to an opposite side to the proximal side, i.e., a patient side. In FIG. 1, the upper side indicates the distal side, and the lower side indicates the proximal side. In each of the drawings, the longitudinal direction of the coil member 2 is indicated by a reference character x. Meanwhile, an inward direction of the coil member 2 refers to a direction, in a radial direction of the coil member 2, toward a center as the longitudinal axis of the coil member 2, and an outward direction of the coil member 2 refers to a radiation direction opposite to the inward direction.

The coil member 2 has a distal end and a proximal end, includes a lumen, is made of a metal, and has flexibility. The coil member 2 is obtained by spirally winding one or more wire materials and is formed as a hollow body. Since the coil member 2 is a hollow body, a tubular member 5 and a stranded wire 30 can be disposed in the lumen of the coil member 2. The cross-sectional shape of each of the wire materials forming the coil member 2 may be any of circular shapes, oblong shapes, polygonal shapes, or combinations thereof. The oblong shapes include an elliptical shape, an oval shape, and a rectangular shape with rounded corners. Each of the wire materials forming the coil member 2 is preferably a flat wire having a rectangular cross-sectional shape.

The density (the winding interval of each wire material) of the coil member 2 is not particularly limited, and the coil member 2 may be of a close-contact winding, a winding at a pitch, or a combination thereof. However, the coil member 2 is preferably formed of a close-contact winding in order to prevent a liquid such as a body fluid from entering the lumen of the coil member 2. A state where wire materials that are adjacent in the longitudinal direction x are in contact with each other is defined as a close-contact winding, and a state where the wire materials are not in contact with each other is defined as a winding at a pitch. The state where the wire materials are not in contact with each other means a state where the wire materials that are adjacent in the longitudinal direction x are apart from each other.

The coil member 2 may be composed of a single layer or may be composed of a plurality of layers. The coil member 2 composed of a plurality of layers can be formed by, for example, forming a first-layer coil through winding of a wire material on a core material and forming a second-layer coil through further winding of a wire material on the first-layer coil.

The coil member 2 is preferably made of a metal and can be made of, for example, stainless steel, carbon steel, aluminum, nickel, copper, titanium, iron, tungsten, gold, silver, or an alloy thereof.

The outer circumferential surface of the coil member 2 is preferably coated with a resin. This enables prevention, to a further extent, of flow of a liquid into the lumen of the coil member 2. Specifically, the coil member 2 is preferably coated with a flexible tube 3 as shown in FIG. 3. The flexible tube 3 can be made of a material which is the same as that of the tubular member 5 described later.

The flexible tube 3 preferably has heat-shrinkable properties. This enables shrinkage of the flexible tube 3 if the coil member 2 is coated with the flexible tube 3 and the flexible tube 3 is heated. Consequently, the flexible tube 3 can be brought into close contact with the coil member 2.

A pair of forceps members 10 is openable and closable, and disposed on the distal side relative to the coil member 2. The pair of forceps members 10 includes, for example, a first forceps member 11 and a second forceps member 12. A proximal end of each forceps member 10 is constantly located on the distal side relative to a distal end 2A of the coil member 2.

The pair of forceps members 10 is preferably supported so as to be pivotable with respect to each other and preferably becomes close to and apart from each other by the pivoting. In FIG. 1 to FIG. 3, each of the first forceps member 11 and the second forceps member 12 includes: a gripping portion A at which an object is picked up; a fulcrum portion B located on the proximal side relative to the gripping portion A and serving as a pivoting fulcrum; and a coupling portion C located on the proximal side relative to the fulcrum portion B and coupled to a corresponding connection member 20.

The gripping portion A is mainly a portion at which an object is picked up. Hereinafter, the gripping portion A of the first forceps member 11 is referred to as a first gripping portion 11A, and the gripping portion A of the second forceps member 12 is referred to as a second gripping portion 12A. The first gripping portion 11A of the first forceps member 11 and the second gripping portion 12A of the second forceps member 12 are preferably disposed so as to face each other. Each gripping portion A preferably has a hollow portion in which at least a part of an object is received. The gripping portion A can be formed in, for example, the shape of a blade, a clip, or a cup having the hollow portion. FIG. 2 shows an example in which the gripping portion A is formed in the shape of a cup. On the first gripping portion 11A and the second gripping portion 12A, respective tooth patterns that are meshed with each other may be formed in order to make it easy to bite into an object. Each tooth pattern may be formed over the entirety of the corresponding gripping portion A and may be formed on the rim of the cup of the gripping portion A as shown in FIG. 2 and FIG. 3. The gripping portion A is preferably provided on the distal side of the forceps member 10.

The fulcrum portion B is a portion serving as a pivoting fulcrum for the forceps member 10. The fulcrum portion B serving as the pivoting fulcrum preferably has an axis in a direction perpendicular to the longitudinal direction x of the coil member 2. In FIG. 2, portions of the first forceps member 11 and the second forceps member 12 on the proximal side relative to the gripping portions A are formed in the shapes of flat plates, distal-side through-holes are respectively formed as the fulcrum portions B in the flat-plate-shape portions, and the center of each distal-side through-hole serves as a pivoting fulcrum. In FIG. 2, only a distal-side through-hole 11B of the first forceps member 11 can be seen. The distal-side through-hole 11B of the first forceps member 11 and the distal-side through-hole of the second forceps member 12 are located so as to overlap with each other in a direction z perpendicular to both the longitudinal direction x of the coil member 2 and a direction y of opening and closing of the forceps 1. If a shaft member 15 is inserted as a rotation shaft into the two distal-side through-holes, the first forceps member 11 and the second forceps member 12 are pivotably supported. The first forceps member 11 and the second forceps member 12 preferably pivot in directions opposite to each other.

As the shaft member 15, for example, a rivet to be riveted, a screw, a bolt, a pin, or the like can be used. In FIG. 2 and FIG. 3, a rivet with a columnar shape having a head portion is inserted as the shaft member 15 into the distal-side through-hole 11B of the first forceps member 11 and the distal-side through-hole of the second forceps member 12. A portion of the rivet on an opposite side to the head portion is riveted and deformed, whereby the first forceps member 11 and the second forceps member 12 are pivotably supported.

In order to ensure a length of each gripping portion A, the corresponding fulcrum portion B is preferably located on the proximal side of the corresponding forceps member 10. In addition, in order to improve the operability of the forceps 1, the fulcrum portion B is preferably located at a portion including the center of the forceps member 10 in the longitudinal direction x of the coil member 2.

The coupling portion C of each forceps member 10 is a portion coupled to the corresponding connection member 20. The coupling portion C may be: a through-hole which is formed in the forceps member 10 and through which the connection member 20 is locked; or a recess which is formed in the forceps member 10 and which is engaged with the connection member 20. In FIG. 2 and FIG. 3, proximal-side through-holes 11C and 12C are formed, as the coupling portions C, in the flat-plate-shape portions of the forceps members 10. A first connection member 21 is locked through the proximal-side through-hole 11C of the first forceps member 11, and a second connection member 22 is locked through the proximal-side through-hole 12C of the second forceps member 12. The plurality of connection members 20 (the first connection member 21 and the second connection member 22) are connected to the stranded wire 30. Thus, when the stranded wire 30 is moved to the distal side or the proximal side, the connection members 20 also move to the distal side or the proximal side. This movement causes the pair of forceps members 10 to pivot with respect to each other about the shaft member 15. Consequently, the forceps 1 can be opened or closed.

From the viewpoint of stably performing an operation of the forceps 1, each coupling portion C is preferably located on the proximal side of the forceps member 10, and, if the length of the forceps member 10 in the longitudinal direction is divided into four equal regions, the coupling portion C is more preferably provided in a region on the most proximal side among the regions.

Each forceps member 10 may be provided with a groove which is located on the proximal side relative to the coupling portion C and in which a part of the corresponding connection member 20 is received. This enables inhibition of the connection member 20 from being displaced from the forceps member 10.

The forceps member 10 and the shaft member 15 can each be made of; a metal such as stainless steel or carbon steel; or a synthetic resin such as a polyamide resin (for example, nylon), a polyolefin resin (for example, polyethylene or polypropylene), a polyester resin (for example, PET), an aromatic polyether ketone resin (for example, PEEK), a polyimide resin, or a fluorine resin (for example, PTFE, PFA, or ETFE). If the endoscope forceps 1 are used as high-frequency hemostasis forceps, an electrically conductive material such as stainless steel needs to be used as the material of the forceps member 10 which is a portion to be brought into contact with a tissue. If the endoscope forceps 1 are used as non-energized forceps (e.g., biopsy forceps), the material of the forceps member 10 does not have to be an electrically conductive material.

Since the connection members 20 are respectively connected to portions on the proximal side of the pair of forceps members 10, the endoscope forceps 1 have two connection members 20. Each connection member 20 is provided for connecting the corresponding forceps member 10 and the stranded wire 30 to each other in the longitudinal direction x of the coil member 2. The connection member 20 may be a linear body such as a single wire or a stranded wire 30, or may have a link mechanism in which a plurality of link plates each having a thin and long shape are combined. A part of the linear body may be bent. Among them, the connection member 20 is preferably a single wire. This enables reduction in friction resistance generated when the connection member 20 comes into contact with the coil member 2. In FIG. 1 to FIG. 3, the first connection member 21 is connected to the first forceps member 11, and the second connection member 22 is connected to the second forceps member 12. At least either of the first connection member 21 and the second connection member 22 may be connected to a positive electrode of a high-frequency power supply. In this case, if a counter electrode plate pasted on the human body is connected to a negative electrode of the high-frequency power supply, high-frequency current can be caused to flow through the forceps members 10, whereby hemostasis can be performed with the forceps 1.

If the connection member 20 is formed of a linear body, the outer diameter of the linear body only has to be set according to the diameter of the forceps channel of the endoscope and can be, for example, not smaller than 0.1 mm and not larger than 3 mm. Further, the outer diameter of the linear body is preferably smaller than the outer diameter of the stranded wire 30. This enables further reduction in the friction resistance generated when the connection member 20 comes into contact with the coil member 2. For fitting along the shape of a body cavity, the length of the connection member 20 in the longitudinal direction x of the coil member 2, i.e., the length of the connection member 20 from a distal end to a proximal end thereof, is preferably not larger than 20 cm and more preferably not larger than 15 cm, and meanwhile, the length is also allowed to be not smaller than 1 cm or not smaller than 5 cm.

If the connection member 20 is a linear body, it is preferable that the linear body is easy to elastically deform, and the linear body is preferably made of, for example, a metal such as: a stainless steel such as SUS303 or SUS304; or Ni—Ti alloy.

The stranded wire 30 is inserted through the lumen of the tubular member 5 and includes a connection portion 31 connected to a proximal end portion of each connection member 20. By moving the stranded wire 30 to the distal side or the proximal side, an opening/closing operation of the forceps 1 can be performed via the connection members 20. In addition, use of the stranded wire makes it easy to transmit torque from the hand side to the forceps member 10 side. The stranded wire 30 extends in the longitudinal direction x of the coil member 2.

The stranded wire 30 can be formed by twisting a plurality of metal element wires or a plurality of metal strands. The stranded wire 30 can be made of, for example, a metal such as: a stainless steel such as SUS303 or SUS304; or carbon steel.

The stranded wire 30 and the two connection members 20 can be connected to each other at the connection portion 31 by, for example, a method such as: mechanical fixation or pressure joining by means of fitting, a screw, riveting, or the like; welding by means of a laser, ultrasonic wave, metal brazing, or the like; or adhesion using an adhesive agent. The stranded wire 30 and the connection members 20 may be directly connected to each other or may be connected to each other via another member. In FIG. 2 and FIG. 3, the stranded wire 30 and the connection members 20 are connected to each other by a connection pipe 40. For example, the stranded wire 30 and the connection members 20 may be connected to each other by disposing the proximal end portions of the two connection members 20 and a distal end portion of the stranded wire 30 in a lumen of one pipe 40 and crimping the pipe 40.

In order to make it easy to perform an operation of moving the stranded wire 30 to the distal side or the proximal side, a proximal end 30B of the stranded wire 30 is preferably located on the proximal side relative to a proximal end 2B of the coil member 2.

The tubular member 5 includes the lumen, is disposed in the lumen of the coil member 2, and prevents contact between the coil member 2 and the stranded wire 30. The tubular member 5 extends along the longitudinal direction x of the coil member 2. The tubular member 5 preferably has flexibility in order to be deformed following the coil member 2. Further, the tubular member 5 preferably has elasticity in order to retain the shape thereof. The tubular member 5 has a longitudinal direction and a radial direction. The longitudinal direction of the tubular member 5 is preferably parallel to the longitudinal direction x of the coil member 2.

The tubular member 5 is preferably a resin tube. This enables reduction in the friction resistance between the coil member 2 and the tubular member 5 and the friction resistance between the tubular member 5 and the stranded wire 30. The resin tube can be produced by, for example, extrusion.

If the tubular member 5 is a resin tube, the tubular member 5 may be composed of a single layer, or at least a part of the tubular member 5 may be composed of a plurality of layers. Alternatively, the tubular member 5 may be obtained by joining together a plurality of tubes in the longitudinal direction.

The tubular member 5 is preferably made of a synthetic resin and more preferably made of, for example, a polyolefin resin such as polyethylene or polypropylene. This enables reduction in the friction resistance between the coil member 2 and the tubular member 5 and the friction resistance between the tubular member 5 and the stranded wire 30.

The tubular member 5 may be obtained by coating, with a resin, at least either of the inner circumferential wall surface or the outer circumferential wall surface of a tubular body which is formed by arranging metal wire materials as single wires or strands in a predetermined pattern. The tubular body formed by arranging the metal wire materials in a predetermined pattern is exemplified by: a tubular body having a mesh structure resulting from mere intersecting or knitting of the metal wire materials; and a coil in which the metal wire materials are wound. The type of the mesh structure is not particularly limited, and the number of turns and the density of the coil are not particularly limited, either. Each of the mesh structure and the coil may be formed such that the density thereof is unchanging over the entirety thereof in the longitudinal direction or such that the density differs depending on the position in the longitudinal direction. The metal wire materials can be made of the same material as that of the coil member 2.

Each connection member 20 and the stranded wire 30 are connected to each other at the connection portion 31. A distal end 5A of the tubular member 5 is located on the distal side relative to the connection portion 31 so as to be present on the proximal side relative to the distal end 2A of the coil member 2. By setting the position of the connection portion 31 and the position of the distal end 5A of the tubular member 5 as described above, contact between the coil member 2 and the stranded wire 30 can be prevented. Thus, an opening/closing operation and a rotation operation of the forceps 1 can be smoothly performed. In addition, disconnection between the connection member 20 and the stranded wire 30 due to contact of the coil member 2 with the connection portion 31 of the stranded wire 30, can also be prevented. Further, since the tubular member 5 is disposed between the coil member 2 and the stranded wire 30, a liquid such as a body fluid is less likely to enter the lumen of the coil member 2, and the liquid can be inhibited from reaching the hand side. By setting the position of the distal end 5A of the tubular member 5 and the position of the distal end 2A of the coil member 2 as described above, the distal end portion of the tubular member 5 is covered with the coil member 2. Thus, even if heat and the like are generated by a high frequency during use of the forceps 1, influence of the heat on the tubular member 5 can be mitigated.

The distal end 5A of the tubular member 5 is preferably located on the distal side relative to a distal end 31A of the connection portion 31. This enables the connection portion 31 to be suitably protected by the tubular member 5.

Although not shown, if a curving portion configured to be curved according to the shape of a body cavity is formed at a distal end portion of the endoscope, the connection portion 31 is preferably located on the proximal side relative to the curving portion. This enables the connection member 20 and the stranded wire 30 to be disconnected from each other by curving of the endoscope. A range of not farther than 10 cm from the distal end of the forceps 1 is preferably set for the curving portion of the endoscope. Therefore, the distal end 31A of the connection portion 31 is preferably located on the proximal side relative to the position that is shifted by 10 cm from the distal end of the forceps 1 to the proximal side.

In the longitudinal direction x of the coil member 2, the length from the distal end 5A of the tubular member 5 to the distal end 2A of the coil member 2 is preferably longer than the length from the distal end 5A of the tubular member 5 to the distal end 31A of the connection portion 31. This enables, even if heat and the like are generated by a high frequency during use of the forceps 1, mitigation of influence of the heat on the tubular member 5.

The outer diameter of the tubular member 5 may be unchanging in the longitudinal direction thereof or may differ depending on the position in the longitudinal direction. As one example, the distal end portion of the tubular member 5 may be provided with a larger-diameter portion having an outer diameter that is larger than the outer diameter of the tubular member 5 at the center position in the longitudinal direction thereof. Provision of the larger-diameter portion in this manner makes it possible to prevent flow of a liquid into an interval between the tubular member 5 and the coil member 2. A maximum inner diameter of the larger-diameter portion of the tubular member 5 is preferably not smaller than 1.1 times, more preferably not smaller than 1.15 times, and further preferably not smaller than 1.2 times the outer diameter of the tubular member 5 at the center position in the longitudinal direction thereof. Meanwhile, in order to inhibit excessive contact between the coil member 2 and the tubular member 5, the maximum inner diameter of the larger-diameter portion of the tubular member 5 is preferably not larger than 1.5 times, more preferably not larger than 1.45 times, and further preferably not larger than 1.4 times the outer diameter of the tubular member 5 at the center position in the longitudinal direction thereof. As another example, a proximal end portion of the tubular member 5 may be provided with a smaller-diameter portion having an outer diameter that is smaller than the outer diameter of the tubular member 5 at the center position in the longitudinal direction thereof. A configuration may be employed in which the tubular member 5 is fixed at the smaller-diameter portion thereof to the stranded wire 30.

The inner diameter of the tubular member 5 may be unchanging in the longitudinal direction thereof or may differ depending on the position in the longitudinal direction. For example, the distal end portion of the tubular member 5 is preferably provided with a smaller-diameter portion having an inner diameter that is smaller than the inner diameter of the tubular member 5 at the center position in the longitudinal direction thereof. This enables prevention of flow of a liquid into the lumen of the tubular member 5. A minimum inner diameter of the smaller-diameter portion is preferably not larger than 0.9 times, more preferably not larger than 0.85 times, and further preferably not larger than 0.8 times the inner diameter of the tubular member 5 at the center position in the longitudinal direction thereof. Meanwhile, in order not to restrict movement of the stranded wire 30 inside the tubular member 5, the minimum inner diameter of the smaller-diameter portion is preferably not smaller than 0.5 times, more preferably not smaller than 0.55 times, and further preferably not smaller than 0.6 times the inner diameter of the tubular member 5 at the center position in the longitudinal direction thereof.

The distal end portion of the tubular member 5 may be provided with a tapered portion that is tapered toward the distal side. The tapered portion only has to be such that at least either of the inner diameter and the outer diameter of the tubular member 5 decreases toward the distal side. This enables prevention of flow of a liquid into the lumen of the tubular member 5. For preferable values of the minimum inner diameter of the tapered portion, refer to the above explanations about the preferable values of the inner diameter of the smaller-diameter portion.

The tubular member 5 is preferably fixed to the stranded wire 30. This makes it possible to maintain the positional relationship, in the longitudinal direction x of the coil member 2, among the distal end 5A of the tubular member 5, the distal end 2A of the coil member 2, and the connection portion 31 of the stranded wire 30. In order to make it easy to maintain this positional relationship, it is preferable that the tubular member 5 does not move to the distal side and the proximal side with respect to the stranded wire 30.

Although the tubular member 5 may be fixed to the stranded wire 30 over the entirety of the stranded wire 30 in the longitudinal direction, the tubular member 5 is preferably fixed to the stranded wire 30 at a part of the stranded wire 30 in the longitudinal direction. In order to enable free rotation of the stranded wire 30, as shown in FIG. 4, the proximal end portion of the tubular member 5 is preferably fixed to the stranded wire 30 and more preferably fixed to a proximal end portion of the stranded wire 30.

A portion, of the proximal end portion of the tubular member 5, that is fixed to the stranded wire 30 is referred to as a proximal-side fixed portion 5C. It is preferable that no portion of the tubular member 5 on the distal side relative to the proximal-side fixed portion 5C is fixed to the stranded wire 30. Further, in order to inhibit contact between the coil member 2 and the proximal-side fixed portion 5C of the tubular member 5, the proximal-side fixed portion 5C of the tubular member 5 is preferably disposed on the proximal side relative to the proximal end 2B of the coil member 2.

The tubular member 5 and the stranded wire 30 can be connected or fixed to each other by, for example, a method such as: mechanical fixation or pressure joining by means of fitting, a screw, riveting, or the like; welding by means of a laser, ultrasonic wave, metal brazing, or the like; or adhesion using an adhesive agent.

A proximal end 5B of the tubular member 5 in the longitudinal direction may coincide with the proximal end 30B of the stranded wire 30 or may be located on the distal side relative to the proximal end 30B of the stranded wire 30.

The tubular member 5 can also be brought into close contact with the stranded wire 30 by, for example, coating the stranded wire 30 with at least a part in the longitudinal direction of the tubular member 5. Meanwhile, in order to improve an effect of inhibiting flow of a liquid from the distal end side of the coil member 2, a space is preferably formed between the coil member 2 and the tubular member 5 or between the tubular member 5 and the stranded wire 30. In order to form such a space, a portion on the distal side of the tubular member 5 (more preferably, the distal end portion of the tubular member 5) is preferably fixed to neither the coil member 2 nor the stranded wire 30.

The tubular member 5 is preferably detached from at least a part in the longitudinal direction x of the coil member 2 and more preferably detached from the entirety in the longitudinal direction x of the coil member 2. This makes it easy to form a space between the tubular member 5 and the coil member 2 and makes it possible to inhibit contact between the coil member 2 and the tubular member 5.

In a cross section of the distal end of the stranded wire 30 perpendicular to the longitudinal direction x of the coil member 2, the spatial cross-sectional area between the coil member 2 and the tubular member 5 is preferably larger than the spatial cross-sectional area between the tubular member 5 and the stranded wire 30. This enables inhibition of contact between the coil member 2 and the tubular member 5.

In the cross section of the distal end of the stranded wire 30 perpendicular to the longitudinal direction x of the coil member 2, the spatial cross-sectional area between the coil member 2 and the tubular member 5 is preferably smaller than the spatial cross-sectional area between the tubular member 5 and the stranded wire 30. This enables prevention of flow of a liquid such as a body fluid into the lumen of the tubular member 5.

The spatial cross-sectional area between the coil member 2 and the tubular member 5 is allowed to be not smaller than 0.05 mm², not smaller than 0.1 mm², not smaller than 0.2 mm², or not smaller than 0.3 mm², and meanwhile, is also allowed to be not larger than 0.7 mm², not larger than 0.6 mm², not larger than 0.5 mm², or not larger than 0.3 mm². The spatial cross-sectional area between the tubular member 5 and the stranded wire 30 is allowed to be not smaller than 0.1 mm², not smaller than 0.2 mm², or not smaller than 0.3 mm², and meanwhile, is also allowed to be not larger than 0.6 mm², not larger than 0.5 mm², or not larger than 0.4 mm².

The proximal end 5B of the tubular member 5 is preferably located on the proximal side relative to the proximal end 2B of the coil member 2. This enables inhibition of contact between the coil member 2 and the stranded wire 30 over a wide range in the longitudinal direction x of the coil member 2.

The endoscope forceps 1 may further include a support member 41 which is disposed on the distal side relative to the distal end 2A of the coil member 2 and which, together with the shaft member 15, supports the pair of forceps members 10 in a pivotable manner. The support member 41 can be formed in, for example, a tubular shape such as the shape of a polygonal tube, a cylinder, or an oblong tube. A proximal end portion of the support member 41 is connected to the distal end portion of the coil member 2. For a method for connecting the support member 41 and the coil member 2 to each other, reference can be made to the explanations about the methods for connecting the connection member 20 and the stranded wire 30 to each other. The support member 41 may be directly connected to the coil member 2 or may be connected to the coil member 2 via another member. In FIG. 3, the support member 41 is connected to the coil member 2 via a connection pipe 42.

If the support member 41 is formed in a tubular shape, the support member 41 may have a first through-hole 41A formed so as to penetrate therethrough between a first position and a second position which are opposed to each other in the circumferential direction thereof, as shown in FIG. 2 and FIG. 3. Alternatively, as another mode, second through-holes may be respectively provided in two protruding portions which extend from a distal end portion of the support member 41 to the distal side and which are opposed to each other. In this case, the shaft member 15 is preferably inserted through either the first through-hole or each second through-hole, and the two distal-side through-holes of the pair of forceps members 10. This enables the pair of forceps members 10 to be pivotably supported by the support member 41, whereby displacement of the rotation shaft at the time of rotation of the first forceps member 11 and the second forceps member 12 is suppressed. The direction of penetration of each of the first through-hole or the second through-hole is preferably different from the longitudinal direction x of the coil member 2 and more preferably parallel to the direction z in which the first forceps member 11 and the second forceps member 12 overlap with each other.

If the support member 41 has a tubular shape, openings 41B may be formed in the circumferential wall of the support member 41, and a part of each forceps member 10 (preferably, a portion on the proximal side of the forceps member 10) may protrude from the corresponding opening 41B. Provision of the openings 41B in this manner enables increase in the range in which the forceps members 10 and the connection members 20 can be moved.

As shown in FIG. 1 and FIG. 4, the endoscope forceps 1 may further include: a protection member 45 connected to the proximal end portion of the coil member 2; and an operation member 50 disposed on the proximal side relative to the coil member 2 and configured to allow an opening/closing operation of the pair of forceps members 10 to be performed through the operation member 50. The operation member 50 may include: a first operation portion 51 rotatably connected to a proximal end portion of the protection member 45; and a second operation portion 52 which is connected to the proximal end portion of the stranded wire 30 and which is moved to the distal side or the proximal side relative to the first operation portion 51. The operation member 50 is a member to be gripped by a user when an opening/closing operation of the forceps 1 is performed. The operation member 50 is connected to a portion on the proximal side of the coil member 2.

In a state where the forceps 1 are opened, the second operation portion 52 is located on the distal side of the first operation portion 51. When the second operation portion 52 is moved to the proximal side with respect to the first operation portion 51, the stranded wire 30 moves to the proximal side. Consequently, the connection members 20 move, whereby the pair of forceps members 10 pivot to come close to each other. Thus, the forceps 1 enter a closed state.

The configuration of the operation member 50 is not particularly limited as long as the first operation portion 51 and the second operation portion 52 move relative to each other. For example, the first operation portion 51 may be a handle body, and the second operation portion 52 may be a slider that is slid with respect to the handle body. Finger hooks may be formed on the first operation portion 51 or the second operation portion 52. As a material of the operation member 50, for example, a synthetic resin such as ABS or polycarbonate, or a foamed plastic such as a polyurethane foam, can be used.

In order to moderate a change in the outer diameter of the forceps 1, the protection member 45 is preferably attached radially outward of the proximal end portion of the coil member 2. The protection member 45 is preferably formed in a tubular shape which has a distal end and a proximal end and which includes a lumen. The protection member 45 may have a portion having an outer diameter that increases toward the proximal side.

The first operation portion 51 may be connected to the protection member 45. This makes it possible to reinforce, from the operation member 50 (the first operation portion 51 and the second operation portion 52), a proximal portion of the endoscope forceps 1 composed of the flexible tube 3, the coil member 2, and the like. The first operation portion 51 may be fixed to the protection member 45 or may be connected to the protection member 45 so as to be movable with respect to the protection member 45. For example, the first operation portion 51 can be connected to the protection member 45 so as to be rotatable with respect to the protection member 45 about the longitudinal direction x of the coil member 2. This enables improvement of the operability of the forceps 1.

A connection portion between the protection member 45 and the first operation portion 51 is preferably located on the distal side relative to a connection portion between the stranded wire 30 and the second operation portion 52. This makes it easy to perform a moving operation of the stranded wire 30 to the distal side or the proximal side. As a method for connection between the coil member 2 and the protection member 45 or between the stranded wire 30 and the second operation portion 52, it is possible to employ, for example: mechanical fixation or pressure joining by means of fitting, a screw, riveting, or the like; welding by means of a laser, ultrasonic wave, metal brazing, or the like; adhesion using an adhesive agent; or the like. These members may be directly connected to each other or may be connected to each other via another member. For example, as shown in FIG. 4, the coil member 2 and the protection member 45 may be connected to each other by fixing, to the protection member 45, a fixation tool 55 connected to the proximal end portion of the coil member 2. Although FIG. 4 shows an example in which the fixation tool 55 is formed in a ring shape, any fixation tool 55 is preferable as long as the fixation tool 55 is attached to the outer circumference of the coil member 2. For example, a cross-sectional shape of the fixation tool 55 perpendicular to the longitudinal direction x of the coil member 2 may be a U shape. For connection between the coil member 2 and the fixation tool 55, the same method as the method for connection between the coil member 2 and the protection member 45 can be employed. For example, if both the coil member 2 and the fixation tool 55 are made of metals, connection therebetween can be made by welding the proximal end portion of the coil member 2 and a distal end portion of the fixation tool 55. If a part of the coil member 2 and at least a part of the fixation tool 55 are fixed so as to be superposed on each other, the fixing strength therebetween can be increased. For connection between the fixation tool 55 and the protection member 45, the same method as the method for connection between the coil member 2 and the protection member 45 can be employed.

When the operation member 50 is rotated with respect to the coil member 2, the rotation angle at the distal end 5A of the tubular member 5 may be smaller than the rotation angle at the proximal end 5B of the tubular member 5. In addition, when the second operation portion 52 is rotated with respect to the coil member 2, the rotation angle at the distal end 5A of the tubular member 5 may be smaller than the rotation angle at the proximal end 5B of the tubular member 5. Further, when the second operation portion 52 is rotated with respect to the first operation portion 51, the distal end 5A of the tubular member 5 does not have to rotate. The distal end 5A of the tubular member 5 becomes difficult to rotate by contact thereof with the coil member 2 or the stranded wire 30.

The first operation portion 51 and the second operation portion 52 may be configured to rotate about the longitudinal direction x of the coil member 2 as if being integral with each other. That is, the first operation portion 51 and the second operation portion 52 do not have to rotate relative to each other about the longitudinal direction x of the coil member 2. If the coil member 2 is fixed to the protection member 45, rotation of the second operation portion 52 with respect to the protection member 45 leads to the same result as the result of rotation of the second operation portion 52 with respect to the coil member 2.

The present application claims the benefit of priority based on Japanese patent application number 2019-094434 filed on May 20, 2019. The entire content of the specification of Japanese patent application number 2019-094434 filed on May 20, 2019 is incorporated herein by reference.

### DESCRIPTION OF REFERENCE SIGNS

1 endoscope forceps
2 coil member
2A distal end
2B proximal end
3 flexible tube
5 tubular member
5A distal end
5B proximal end
5C proximal-side fixed portion
10 forceps member
11 first forceps member
11A first gripping portion
11B distal-side through-hole
11C proximal-side through-hole
12 second forceps member
12A second gripping portion
12C proximal-side through-hole
15 shaft member
20 connection member
21 first connection member
22 second connection member
30 stranded wire
30B proximal end
31 connection portion
31A distal end
40 pipe
41 support member
42 pipe
45 protection member
50 operation member
51 first operation portion
52 second operation portion
55 fixation tool
A gripping portion
B fulcrum portion
C coupling portion

1. An endoscope forceps comprising:
a coil member having a distal end and a proximal end and including a lumen, the coil member made of a metal and having flexibility;
a tubular member including a lumen and disposed in the lumen of the coil member;
a pair of forceps members disposed on a distal side relative to the coil member, and openable and closable;
connection members respectively connected to portions on a proximal side of the pair of forceps members; and
a stranded wire inserted through the lumen of the tubular member and including a connection portion connected to proximal end portions of the connection members, such that the pair of forceps members are operable by moving the stranded wire, wherein
the distal end of the coil member is located, in a longitudinal direction of the coil member, between a distal end of the tubular member and the connection portion at which each connection member is connected to the stranded wire.

2. The endoscope forceps according to claim 1, wherein
the tubular member is made of a synthetic resin.

3. The endoscope forceps according to claim 1, wherein
a proximal end of the tubular member is located on the proximal side relative to the proximal end of the coil member.

4. The endoscope forceps according to claim 1, wherein
a proximal end portion of the tubular member is fixed to the stranded wire.

5. The endoscope forceps according to claim 1, wherein
each connection member is a single wire.

6. The endoscope forceps according to claim 1, wherein
in a cross section of a distal end of the stranded wire perpendicular to the longitudinal direction of the coil member, a spatial cross-sectional area between the coil member and the tubular member is larger than a spatial cross-sectional area between the tubular member and the stranded wire.

7. The endoscope forceps according to claim 1, wherein
in a cross section of a distal end of the stranded wire perpendicular to the longitudinal direction of the coil member, a spatial cross-sectional area between the coil member and the tubular member is smaller than a spatial cross-sectional area between the tubular member and the stranded wire.

8. The endoscope forceps according to claim 1, wherein
a distal end portion of the tubular member is provided with a smaller-diameter portion having an outer diameter that is smaller than an outer diameter of the tubular member at a center position in a longitudinal direction thereof.

9. The endoscope forceps according to claim 1, wherein
a distal end portion of the tubular member is provided with a tapered portion that is tapered toward the distal side.

10. The endoscope forceps according to claim 1, wherein
the stranded wire is made of a metal.

11. The endoscope forceps according to claim 1, further comprising:
a protection member connected to a proximal end portion of the coil member; and
an operation member disposed on the proximal side relative to the coil member and configured to allow an opening/closing operation of the pair of forceps members to be performed through the operation member, wherein
the operation member includes
a first operation portion rotatably connected to a proximal end portion of the protection member, and
a second operation portion connected to a proximal end portion of the stranded wire and configured to be moved relative to the first operation portion, and
the protection member is connected to the first operation portion at the distal side relative to a connection portion at which the stranded wire is connected to the second operation portion.

12. The endoscope forceps according to claim 11, wherein
the operation member, the tubular member and the coil member are arranged so that when the operation member is rotated with respect to the coil member, a rotation angle at the distal end of the tubular member is smaller than a rotation angle at a proximal end of the tubular member.
